(19) United States
(12) Patent Application Publication
(10) Pub. No.: US 2006/0135685 A1
(43) Pub. Date: Jun. 22, 2006

(54) Ester mixtures

(76) Inventors: Jan-Gerd Hansel, Koln (DE); Eberhard Kuckert, Leverkusen (DE); Ulrich Feldhues, Gladbach (DE)

Correspondence Address: LANXESS CORPORATION, 111 RIDC PARK WEST DRIVE, PITTSBURGH, PA, 15275-1112, US

(21) Appl. No.: 11/301,681

(22) Filed: Dec. 13, 2005

(30) Foreign Application Priority Data
Dec. 14, 2004 (DE) 10 2004 06 043.0

(51) Int. Cl. C09D 5/02; C07C 69/76

(52) U.S. Cl. 524/556; 524/567; 524/565; 560/100; 560/112

(57) ABSTRACT

The present invention relates to ester mixtures comprising trimethylolalkane esters of aromatic carboxylic acids, trimethylolalkane esters of aliphatic carboxylic acids and trimethylolalkane esters both of aromatic and aliphatic carboxylic acids, to a process for their preparation, and to their use as plasticizers for polymers.

The present invention relates to ester mixtures comprising trimethylolalkane esters of aromatic carboxylic acids, trimethylolalkane esters of aliphatic carboxylic acids and trimethylolalkane esters both of aromatic and aliphatic carboxylic acids, to a process for their preparation, and to their use as plasticizers for polymers.

Plasticizers are substances which are added to brittle and hard polymers, for example polyvinyl chloride (PVC), in order to impart to them properties desirable for processing and use, such as flexibility and tensility.

The important substance properties of plasticizers for the application are described, for example, in David F. Cadogan, Christopher J. Howick: “Plasticizers”, Ullmann's Encyclopedia of Industrial Chemistry, Electronic Release, 6th ed., chap. 1-6, Wiley-VCH, Weinheim 2003 and L. Meier: “Plasticizers”, in R. Gächter, H. Müller (Ed.): Taschenbuch der Kunststoffadditive [Handbook of plastics additives], 3rd edition, p. 357-p. 382, Hanser Verlag, Munich 1990. In general, liquid plasticizers are used. They preferably have a viscosity of below 100 000 mPa·s, a dissolution temperature in polyvinyl chloride of below 170° C. and an acid number of below 1 mg KOH/g.

For the use of a substance as a plasticizer, it is also important that it remains substantially permanently within the polymer plasticized with it. Many plasticizers tend to migrate into substances in contact with the plasticized polymer, for example other polymers. Lubricants or blowing agents and also soap solutions can leach out plasticizers. Plasticizers with insufficient compatibility with the polymer to be plasticized can be deposited on the surface after processing and form an undesired, greasy film. Finally, plasticizers, owing to their volatility, can evaporate out of a plasticized polymer formulation. This leads firstly to undesired embrittlement of the polymer formulation and secondly to the likewise undesired precipitation of the plasticizer on cold surfaces. All of these phenomena occur to a particular degree when an object produced from plasticized plastic is exposed to elevated temperatures for a prolonged period. One example of these so-called high-temperature applications is that of cable sheathing which is used in the engine compartment of an automobile.

Preference is thus given to using plasticizers having low volatility, good compatibility and low migration tendency.

In addition to phosphoric esters and sulphonic esters, especially the alkyl esters of carboxylic acids have favourable substance properties for use as plasticizers. The technically relevant plasticizers and their use are known and are described, for example, in David F. Cadogan, Christopher J. Howick: “Plasticizers”, Ullmann's Encyclopedia of Industrial Chemistry, Electronic Release, 6th ed., chap. 1-6, Wiley-VCH, Weinheim 2003 and L. Meier: “Plasticizers”, in R. Gächter, H. Müller (Ed.): Taschenbuch der Kunststoffadditive, 3rd edition, p. 341 ff., Hanser, Munich 1990. The volatility of substances generally decreases with increasing molecular weight. Therefore, the esters used are usually not simple esters, but rather, owing to their higher molecular weights, preferably esters of polybasic carboxylic acids with monohydric alcohols, esters of monobasic carboxylic acids with polyhydric alcohols or esters of polybasic carboxylic acids with polyhydric alcohols. The latter group of esters comprises oligomeric or polymeric esters which, owing to their high viscosities, are more difficult to process than the former two groups of low molecular weight esters.

Esters of di- and tribasic carboxylic acids with monohydric alcohols are used the most frequently as plasticizers. Examples thereof are the phthalic esters, for example di(2-ethylhexyl) phthalate (DEHP), or trimellitic esters, for example trioctyl trimellitate (TOTM). Owing to their comparatively high volatility for high-temperature applications, diesters such as di(2-ethylhexyl) phthalate are generally unsuitable. The corresponding triester of the tribasic trimellitic acid, trioctyl trimellitate, is established as a low-volatility plasticizer for high-temperature applications. However, trioctyl trimellitate is more difficult to process, less readily available and distinctly more expensive than di(2-ethylhexyl) phthalate, and therefore cannot be used for many applications.

Some phthalic esters, for example di(2-:ethylhexyl) phthalate (DEHP) have in recent times come under suspicion of being harmful to health. Thus, according to the Dangerous Substances Directive 67/548/EEC, they have to be classified in the EU as possibly impairing fertility and possibly having developmental toxicity.

Esters of monobasic acids with dihydric alcohols can likewise be used advantageously as plasticizers. For example, U.S. Pat. No. 2,956,978 B1 describes dibenzoates of various glycols as plasticizers. As U.S. Pat. No. 6,184,278 B1 teaches, some of these dibenzoates, for example ethylene glycol dibenzoate, diethylene glycol dibenzoate or triethylene glycol dibenzoate, have the disadvantage that they are solid at 25° C.;

U.S. Pat. No. 2,585,448 B1, U.S. Pat. No. 3,370,032 B1 and US 2003/0023112 A1 describe mixed esters which are prepared by reaction of diols with a mixture of aliphatic and aromatic carboxylic acids. However, the relatively high volatility of the diol esters excludes them from many applications.

DE 2 318 411 A1 proposes, as a plasticizer for hot-melt adhesive preparations, the substance trimethylolpropane tribenzoate. Since this substance is a solid, it is generally unsuitable as a plasticizer.

U.S. Pat. No. 3,072,591 describes esters of polymethylolalkanes, for example trimethylolpropane, of at least one aromatic carboxylic acid and aliphatic carboxylic acids of at least 6 carbon atoms, which can be used as plasticizers for vinyl chloride polymers. These esters are known as “mixed esters” in which the same molecule contains both radicals of the aromatic carboxylic acids and of the aliphatic carboxylic acids. One example thereof is trimethylolpropane dibenzoate monolaurate. These “mixed esters” are explicitly differentiated from physical mixtures of esters which contain only aromatic or only aliphatic acid radicals (see U.S. Pat. No. 3,072,591, column 5, lines 15-17). The synthesis of the “mixed esters” claimed in U.S. Pat. No. 3,072,591 entails a two-stage synthetic process (see U.S. Pat. No. 3,072,591, column 5, lines 8-15). This process has the disadvantage that it is time-consuming and laborious, since it is necessary to work at two different reaction temperatures. Owing to this preparation process, it is difficult to prepare the “mixed esters” on the industrial scale.

U.S. Pat. No. 3,894,959 describes esters which a prepared by esterification of a mixture consisting of 1 to 50 mol % of an aromatic monocarboxylic acid and 99 to 50 mol % of an aliphatic monocarboxylic acid with a monohydric alcohol having 3 to 6 carbon atoms and 2 to 4 hydroxyl groups. These substances are proposed as electrically insulating oils. Use as a plasticizer is not mentioned.

U.S. Pat. No. 3,929,201 describes esters which are obtained by esterification of monocarboxylic acids with triethanolmethane (3-(2-hydroxyethyl)pentane-1,5-diol). Instead of a monocarboxylic acid, it is also possible to use a mixture of different monocarboxylic acids, for example a mixture of an aromatic and an aliphatic carboxylic acid. The claimed esters are useful as plasticizers for polyvinyl chloride. However, they have the serious disadvantage that the triethanolmethane raw material needed for their preparation cannot be prepared industrially in a simple manner and is also not commercially available.

WO 02/053635 describes mixtures of esters of trimethylolpropane, benzoic acid and 2-ethylhexanoic acid which are used as plasticizers, preferably for polyvinyl chloride. However, the use of ethylhexanoic acid is, according to the information of W. J. Scott, M. D. Collins, H. Nau; Environmental Health Supplements, Volume 102, Number S11, 1994, toxicologically controversial and should be avoided.

It is therefore an object of the present invention to provide plasticizers for polymers which can be prepared in a simple manner on the industrial scale and have favourable processing properties, are liquid at room temperature and also remain liquid in the course of prolonged storage, feature low volatility and high thermal stability, and comprise a minimum of toxicologically controversial substances.

This object is achieved by ester mixtures containing

(A) 5-22% by weight of a compound of the general formula (I)

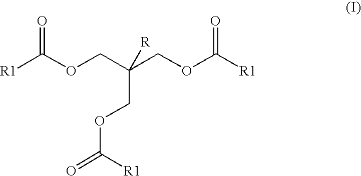

in which
R is H or a C₁- to C₄-alkyl chain and
R¹ is a C₆- to C₁₄-aryl radical optionally substituted by one to three C₁- to C₄-alkyl radicals,

(B) 26-44% by weight of a compound of the general formula (II) 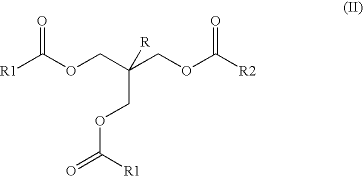
in which
R and R′ are each as defined above and
R² is a straight-chain or branched C₁₁- to C₂₁-alkyl radical,

(C) 28-45% by weight of a compound of the general formula (III) 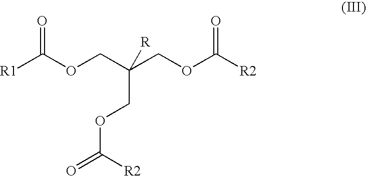
in which
R, R¹ and R² are each as defined above and

(D) 6-25% by weight of a compound of the general formula (IV) 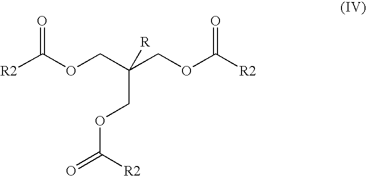
in which
R and R² are each as defined above.

The R radical derives preferably from trimethylolalkanes of the general formula (V)

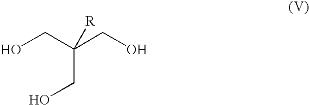

in which

R is H or a C₁- to C₄-alkyl chain.

Examples thereof are trimethylolethane (R═CH₃) or trimethylolpropane (R═CH₂CH₃). The inventive ester mixtures may comprise esters of a plurality of different trimethylolalkanes. The R radical is particularly derived from trimethylolpropane.

The R¹ radical derives preferably from aromatic monocarboxylic acids such as benzoic acid, o-toluic acid, m-toluic acid, p-toluic acid, 4-tert-butylbenzoic acid, 1-naphthoic acid or 2-naphthoic acid, in particular from benzoic acid.

The R² radical derives preferably from aliphatic monocarboxylic acids such as lauric acid, myristic acid, palmitic acid, stearic acid, oleic acid, arachic acid or behenic acid, in particular from lauric acid.

The inventive esters preferably have an acid number of less than or equal to 1 mg KOH/g. They preferably have an acid number of less than or equal to 0.5 mg KOH/g (see page 14).

The invention also encompasses a process for preparing the trimethylolalkane esters to be used in the inventive ester mixtures, characterized in that

a) one or more trimethylolalkanes are esterified with

b) 108 to 180 mol % (based on 100 mol % of trimethylolalkane) of one or more aliphatic C₁₂- to C₂₂-monocarboxylic acids or aliphatic C₁₂- to C₂₂-monocarboxylic acid derivatives and

c) 120 to 300 mol % (based on 100 mol % of trimethylolalkane) of one or more aromatic C₇- to C₁₅-monocarboxylic acids or aromatic C₇- to C₁₅-monocarboxylic acid derivatives

d) at temperatures between 150° C. and 300° C.

In a preferred embodiment of the process according to the invention, the esterification can be carried out

e) with the aid of catalysts and/or

f) with removal of volatile by-products of the esterification, for example water.

As described below, the reactants used react in such a way that the acyl radicals are distributed between the originally present hydroxyl groups. As a consequence of this, the reaction products, after full esterification, are always mixtures of components I to IV, which is why the esterification process can at the same time be regarded as a process for preparing mixtures of components I to IV.

The carboxylic acids or carboxylic acid derivatives can be esterified simultaneously or successively. It is preferably effected simultaneously in such a way that a mixture of the carboxylic acids or carboxylic acid derivatives is used in the esterification.

The esterification reactions can be accelerated with the aid of customary catalysts, for example titanium(IV) isopropoxide, titanium(IV) butoxide, tin(II) 2-ethylhexanoate, and/or of entraining agents, for example toluene or xylene. However, the inventive ester mixtures can also be prepared by the reaction of the above-described trimethylolalkanes with derivatives of the carboxylic acids used, for example carboxylic esters, carboxylic anhydrides or carbonyl halides. These and further methods are known to those skilled in the art and are described, for example, in W. Riemenschneider: “Esters, Organic”, Ullmann's Encyclopedia of Industrial Chemistry, Electronic Release, 6th edition, ch. 5, Wiley-VCH, Weinheim 2003. The esterification can be carried out up to full conversion of all hydroxyl groups present in the reaction mixture, or else terminated at incomplete conversion. Preference is given to attaining a conversion of greater than 90% of the hydroxyl groups present. In addition to the actual synthesis of the esters, their preparation can also include one or more workup steps, for example washing with water or aqueous solutions, bleaching, distillation, drying, filtration and the like.

The sum of the carboxylic acids used may be smaller than, equal to or greater than 300 mol %, based on 100 mol % of trimethylolalkane. Preference is given to using 300 to 350 mol % of carboxylic acid mixture based on 100 mol % of trimethylolalkane.

After the end of the reaction, a residue of unconverted carboxylic acids may remain in the reaction mixture. This is to be expected in particular when more carboxylic acids are used than correspond to 300 mol % in relation to 100 mol % of trimethylolalkane, or when the esterification is terminated at incomplete conversion. In the inventive preparation process, a residue of unconverted carboxylic acids is optionally removed from the reaction mixture by one or more of the above-listed workup steps.

The aromatic C₇- to C₁₅-monocarbbxylic acids or aromatic C₇- to C₁₅-monocarboxylic acid derivatives used are preferably benzoic acid, o-toluic acid, m-toluic acid, p-toluic acid, 4-tert-butylbenzoic acid, 1-naphthoic acid and/or 2-naphthoic acid, or derivatives of these acids or mixtures thereof. Preference is given to using benzoic acid.

The aliphatic C₁₂- to C₂₂-monocarbbxylic acids or aliphatic C₁₂- to C₂₂-monocarboxylic acid derivatives used are preferably lauric acid, myristic acid, palmitic acid, stearic acid, oleic acid, arachic acid and/or behenic acid, or derivatives of these acids or mixtures thereof. Preference is given to using lauric acid and/or palmitic acid.

Very particular preference is given to using
trimethylolpropane as the trimethylolalkane;
benzoic acid as the aromatic C₇- to C₁₅-monocarboxylic acid; and
lauric acid as the aliphatic C₁₂- to C₂₂-monocarboxylic acid.

In the inventive preparation process, the reactants used react in such a way that the acyl radicals are distributed between the originally present hydroxyl groups. This distribution may, for example, be random. As a consequence of this distribution, the reaction products, after full esterification, are always mixtures of the above-described components I to IV.

The invention also encompasses the use of the ester mixtures as plasticizers for polymers such as polyvinyl chloride, vinyl chloride-based copolymers, polyvinylidene chloride, polyvinyl acetals, polyacrylates, polyamides, polylactides; cellulose and its derivatives, rubber polymers such as acrylonitrile-butadiene rubber, hydrogenated acrylonitrile-butadiene rubber, chloroprene rubber, chlorinated polyethylene, chlorosulphonyl polyethylene, ethylene-propylene rubber, acrylate rubber and/or epichlorohydrin rubber. Preference is given to polyvinyl chloride.

In this case, the polyvinyl chloride is prepared preferably by homopolymerization from vinyl chloride by the methods known to those skilled in the art, such as suspension, emulsion or bulk polymerization. The inventive ester mixtures are preferably used in mixtures with 20 to 99% polyvinyl chloride, preferably 45 to 95% polyvinyl chloride, more preferably 50 to 90% polyvinyl chloride. These mixtures are known as soft polyvinyl chloride and may, in addition to the inventive ester mixtures and polyvinyl chloride, also comprise other suitable additives. For example, stabilizers, lubricants, fillers, pigments, flame retardants, light stabilizers, blowing agents, polymeric processing assistants, impact modifiers, optical brighteners, antistats and/or biostabilizers may be present.

The present invention also relates to polymers which comprise the inventive mixtures.

These polymers to be synthesized in accordance with the invention by means of the ester mixtures also preferably comprise additives such as stabilizers, lubricants, fillers, pigments, flame retardants, light stabilizers, blowing agents, polymeric processing assistants, impact modifiers, optical brighteners, antistats and/or biostabilizers, and also mixtures thereof.

Some suitable additives will be described in detail below. However, the examples adduced do not constitute any restriction of the inventive mixtures, but rather serve merely for illustration. All content data are % by weight.

Stabilizers neutralize the hydrochloric acid released during and/or after the processing of the polyvinyl chloride. In a preferred embodiment of the inventive ester mixtures and in the polymers to be prepared therefrom, useful stabilizers are all customary polyvinyl chloride stabilizers in solid and liquid form, for example customary epoxy/zinc, Ca/Zn, Ba/Zn, Pb or Sn stabilizers, and also acid-binding sheet silicates such as hydrotalcite. The inventive ester mixtures may be used in mixtures with a content of stabilizers of 0.05 to 7%, preferably 0.1 to 5%, more preferably 0.2 to 4% and in particular 0.5 to 3%.

Lubricants should be effective between the polyvinyl chloride particles and counteract frictional forces in the course of mixing, plasticizing and reshaping. In a preferred embodiment, the lubricants present in the inventive mixtures may be all lubricants which are customary for the processing of polymers. For example, useful lubricants are hydrocarbons such as oils, paraffins and PE waxes, fatty alcohols having 6 to 20 carbon atoms, ketones, carboxylic acids such as fatty acids and montanic acids, oxidized PE wax, metal salts of carboxylic acids, carboxamides and carboxylic esters, for example with the alcohols ethanol, fatty alcohols, glycerol, ethanediol, pentaerythritol and long-chain carboxylic acids as the acid component. The inventive ester mixtures may be used in mixtures having a content of lubricants of 0.01 to 10%, preferably 0.05 to 5%, more preferably 0.1 to 3% and in particular 0.2 to 2%.

Fillers influence in particular the compressive strength, tensile strength and flexural strength, and also the hardness and heat distortion resistance, of plasticized polyvinyl chloride or PVB in a positive way. In the context of the invention, the mixtures, in a preferred embodiment, may also comprise fillers, for example carbon black and other inorganic fillers, such as natural calcium carbonates, for example chalk, limestone and marble, synthetic calcium carbonates, dolomite, silicates, silica, sand, diatomaceous earth, aluminium silicates, such as kaolin, mica and feldspar. The fillers used are preferably calcium carbonates, chalk, dolomite, kaolin, silicates, talc or carbon black. The inventive ester mixtures may be used in mixtures having a content of fillers of 0.01 to 80%, preferably 0.1 to 60%, mote preferably 0.5 to 50% and in particular 1 to 40%.

The mixtures formulated with the inventive ester mixtures may, in a preferred embodiment, also comprise pigments in order to adjust the resulting product to different possible uses. In the context of the present invention, both inorganic pigments and organic pigments may be used. The inorganic pigments used may, for example, be cadmium pigments such as CdS, cobalt pigments such as CoO/Al₂O₃, and chromium pigments, for example Cr₂O₃. The organic pigments used may, for example, be monoazo pigments, condensed azo pigments, azomethine pigments, anthraquinone pigments, quinacridonies, phthalocyanine pigments, dioxazine pigments and aniline pigments. The inventive ester mixtures may be used in mixtures having a content of pigments of 0.01 to 10%, preferably 0.05 to 5%, more preferably 0.1 to 3% and in particular 0.5 to 2%.

In order to reduce the flammability and the evolution of smoke in the course of burning, the inventive mixtures, in a preferred embodiment, may also comprise flame retardants. The flame retardants used may, for example, be antimony trioxide, phosphate esters, chloroparaffin, aluminium hydroxide, boron compounds, molybdenum trioxide, ferrocene, calcium carbonate or magnesium carbonate. The inventive ester mixtures may be used in mixtures having a content of flame retardant of 0.01 to 30%, preferably 0.1 to 25%, more preferably 0.2 to 20% and in particular 0.5 to 15%.

In order to protect articles which have been produced from a mixture comprising the inventive ester mixtures from damage in the surface region by the influence of light, the mixtures may, in a preferred embodiment, also comprise light stabilizers. In the context of the present invention, it is possible, for example, to use hydroxybenzophenones or hydroxyphenylbenzotriazoles. The inventive ester mixtures may be used in mixtures having a content of light stabilizers of 0.01 to 7%, preferably 0.1 to 5%, more preferably 0.2 to 4% and in particular 0.5 to 3%.

The polymers comprising the inventive ester mixtures present therein may, in a preferred embodiment, also comprise further plasticizers such as monoalkyl esters of benzoic acid, benzoic diesters of mono-, di-, tri- or polyalkylene glycols, esters of monocarboxylic acids with polyols, dialkyl esters of aliphatic dicarboxylic acids, dialkyl esters of aromatic dicarboxylic acids, trialkyl esters of aromatic tricarboxylic acids, phenyl esters of alkanesulphonic acids, alkyl or aryl esters of phosphoric acid, polyesters of dicarboxylic acids, and also mixtures thereof. The polymers preferably comprise trialkyl esters of aromatic tricarboxylic acids as further plasticizers.

Examples of further plasticizers are
the monoalkyl esters of benzoic acid, for example isononyl benzoate,
the benzoic diesters of mono-, di-, tri- or polyalkylene glycols, for example propylene glycol dibenzoate, diethylene glycol dibenzoate, dipropylene glycol dibenzoate, triethylene glycol dibenzoate or polyethylene glycol dibenzoate, and in particular mixtures thereof,
esters of monocarboxylic acids with polyols, for example esterification products obtainable from benzoic acid, butyric acid and glycerol, esterification products obtainable from benzoic acid, lauric acid and glycerol, esterification products obtainable from benzoic acid, lauric acid and diethylene glycol, or esterification products obtainable from benzoic acid, lauric acid and neopentyl glycol,
the dialkyl esters of aliphatic dicarboxylic acids, for example di(2-ethylhexyl) adipate, diisononyl adipate, di(2-ethylhexyl) sebacate, di(2-ethylhexyl) azelate, diisononyl cyclo-hexane 1,2-dicarboxylate,
the dialkyl esters of aromatic dicarboxylic acids, for example di(2-ethylhexyl) phthalate, diisononyl phthalate, diisodecyl phthalate, benzyl butyl phthalate, benzyl isooctyl phthalate, benzyl isononyl phthalate,
the trialkyl esters of aromatic tricarboxylic acids, for example trioctyl trimellitate,
the phenyl esters of alkanesulphonic acids, for example the product Mesamoll® from LANXESS Deutschland GmbH,
the alkyl or aryl esters of phosphoric acid, for example tri(2-ethylhexyl) phosphate, diphenyl 2-ethylhexyl phosphate, diphenyl cresyl phosphate or tricresyl phosphate,
polyesters which can be prepared, for example, from dicarboxylic acids such as adipic acid or phthalic acid, and diols such as 1,2-propanediol, 1,3-butanediol, 1,4-butanediol or 1,6-hexanediol.

In the context of the invention, the inventive ester mixtures, in a preferred embodiment, may also be used in mixtures which comprise further polymers selected from the group consisting of homo- and copolymers based on ethylehe, propylene, butadiene, vinyl acetate, glycidyl acrylate, glycidyl methacrylate, acrylates and methacrylates having alcohol components of branched or unbranched C₁- to C₁₀-alcohols, styrene or acrylonitrile. Examples include polyacrylates having identical or different alcohol radicals from the group of the C₄- to C₈-alcohols, particularly of butanol, hexanol, octanol and 2-ethylhexanol, polymethyl methacrylate, methyl methacrylate-butyl acrylate copolymers, methyl methacrylate-butyl methacrylate copolymers, ethylene-vinyl acetate copolymers, chlorinated polyethylene, nitrile rubber, acrylonitrile-butadiene-styrene copolymers, ethylene-propylene copolymers, ethylene-propylene-diene copolymers, styrene-acrylonitrile copolymers, acrylonitrile-butadiene rubber, styrene-butadiene elastomers and methyl methacrylate-styrene-butadiene copolymers.

The mixtures prepared with the inventive ester mixtures are, for example, useful for the production of pipe lines, cables, wire sheathing, in interior design, in vehicle and furniture construction, in floor coverings, medical articles, food packaging, gaskets, films, composite films, films for composite safety glass, in particular for the vehicles sector and the architecture sector, synthetic leather, toys, packaging containers, adhesive tape films, clothing, coatings, and also fibres for fabrics.

The inventive ester mixtures have good processability and low volatility. Soft polyvinyl chloride articles produced with the inventive ester mixtures feature in particular very good thermostability and are characterized by a low weight loss in the course of thermal ageing in a forced-air oven, and a high HCl stability in the Congo Red test.

The invention will be illustrated in detail with reference to the examples which follow, without any intention that this should bring about a restriction of the invention.

It will be understood that the specification and examples are illustrative but not limitative of the present invention and that other embodiments within the spirit and scope of the invention will suggest themselves to those skilled in the art.

### EXAMPLES

The parts specified are by weight.

Experimental Method

268.4 parts of trimethylolpropane, 464.1 parts of benzoic acid as the aromatic monocarboxylic acid and 440.7 parts of lauric acid as the aliphatic monocarboxylic acid and 120 parts of xylene as the entraining agent were melted under a gentle nitrogen stream in a four-necked flask with stirrer, contact thermometer, water separator, reflux condenser and hotplate with regulator. 3.4 parts of titanium tetra(isopropoxide) were added as the catalyst and the mixture was boiled at 190° C. with stirring for 25.5 h. After this time, 103 parts of water had separated out. The volatile constituents were drawn off at 190° C. and 3 mbar within 3 h. The reaction product was isolated and the acid number determined.

### Examples 1 to 4 and Noninventive Comparative Examples C1 to C3

The inventive compounds 1 to 4 and the noninventive compounds C1 to C3 were prepared by the above method using the starting materials listed in Table 1. The substance C1 is a solid. It was recrystallized from ethanol and melts at 82° C.

Composition of the Ester Mixtures

The composition of the ester mixtures was determined by proton NMR spectroscopy. To this end, the signals of the CH₂ groups of the trimethylolpropane radicals were integrated and the relative molar fractions of the individual components were calculated from the integrals. With the aid of the molar masses, the fractions by weight of the components can be calculated from the molar fractions and are specified in Table 1 as percentages by weight (% by wt.).

Table 1 shows that ester mixtures in the inventive composition can be prepared in a simple manner by the inventive preparation process with suitable selection of the ratios of the starting materials relative to one another.

TABLE 1
Inventive Examples 1 to 4 and noninventive Examples C1 to C3

| | Trimethylol- | Acid(s) | benzoic acid | lauric acid | Component I²⁾ | Component II³⁾ | Component III⁴⁾ | Component IV⁵⁾ |
|---|---|---|---|---|---|---|---|---|
| Example | propane (parts) | (parts) | mol %¹⁾ | mol %¹⁾ | % by wt. | % by wt. | % by wt. | % by wt. |
| 1 | (268.4) | Benzoic acid (464.1), | 190 | 110 | 21 | 43 | 30 | 7 |
| | | Lauric acid (440.7) | | | | | | |
| 2 | (268.4) | Benzoic acid (439.6), | 180 | 120 | 17 | 42 | 33 | 8 |
| | | Lauric acid (480.8) | | | | | | |
| 3 | (268.4) | Benzoic acid (415.2), | 170 | 130 | 13 | 39 | 38 | 10 |
| | | Lauric acid (520.8) | | | | | | |
| 4 | (268.4) | Benzoic acid (366.4), | 150 | 150 | 10 | 35 | 40 | 15 |
| | | Lauric acid (601.0) | | | | | | |
| C1 | (134.2) | Benzoic acid (366.4) | 300 | 0 | 99 | 0 | 0 | 0 |
| DE 2 318 | | | | | | | | |
| 411 A1 | | | | | | | | |
| C2 | (268.4) | Benzoic acid (495.8), | 203 | 101 | 25 | 44 | 26 | 5 |
| | | Lauric acid (406.6) | | | | | | |
| C3 | (134.2) | Benzoic acid (146.5), | 120 | 230 | 2 | 18 | 44 | 36 |
| | | Lauric acid (460.7) | | | | | | |

¹⁾in the reaction mixture based on 100 mol % of trimethylolpropane
²⁾Component I: Trimethylolpropane tribenzoate
³⁾Component II: Trimethylolpropane dibenzoate monolaurate
⁴⁾Component III: Trimethylolpropane monobenzoate dilaurate
⁵⁾Component IV: Trimethylolpropane trilaurate

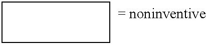

Physical Properties of the Ester Mixtures

The physical data important for ester mixtures (see Table 2) were determined by the following methods:

Viscosity: to DIN 53015 (2001) by means of Höppler falling-ball viscometer

Pour Point: to DIN ISO 3016 (1982)

Acid Number: to EN ISO 3682 (1998)

TABLE 2
Physical properties of the ester mixtures

| | Acid number | Viscosity | Pour point | Dissolution temperature | Time until crystallization |
|---|---|---|---|---|---|
| Example | mg KOH/g | mPa · s (23° C.) | ° C. | ° C. | at 4° C., d |
| 1 | 0.8 | 463 | −27 | 151 | >60 |
| 2 | 0.6 | 373 | −27 | 152 | >60 |
| 3 | 0.4 | 301 | −34 | 154 | >60 |
| 4 | 0.9 | 223 | −27 | 161 | >60 |
| C1 | 0.3 | Solid | Solid | n.d. | Solid |
| C2 | 1.6 | 565¹⁾ | −26¹⁾ | 146 | 4 |
| C3 | 6.6 | 97 | −13 | 200 | >60 |

¹⁾after storage at room temperature for three weeks a large amount of a crystalline precipitate forms

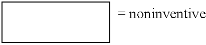

For the handling and processing of ester mixtures as plasticizers, their viscosity and pour point are important characteristic parameters;.

Commercial plasticizers are liquids having viscosities between about 10 mPa·s and more than 10 000 mPa·s (see, for example, L. Meier: “Weichmacher”, in R. Gächter, H. Müller (Ed.): Taschenbuch der Kunststoffadditive, 3rd edition, p. 383- p. 425, Hanser Verlag, Munich 1990). The examples cited lie within this preferred viscosity range.

The pour point indicates the lowest temperature at which a liquid is still free-flowing. The pour points of the examples cited are so low that the substances retain unrestricted free flow at customary processing temperatures of above 15° C.

Crystallization Tendency

The processors of soft polyvinyl chloride are equipped for the use of liquid plasticizers. Full or partial crystallization of an originally liquid plasticizer, for example during storage, is undesired, since the redissolution or melting and homogenization constitute additional working steps.

The substance C1 has been proposed in DE 2 318 411 A1 as a plasticizer for hot-melt adhesive preparations. Since C1 is a solid, it is unsuitable for the usual processing to give soft polyvinyl chloride.

U.S. Pat. No. 3,072,591 B1 proposes the substance trimethylolpropane dibenzoate monolaurate as a plasticizer for polyvinyl chloride. A substance mixture C2 in which benzoyl and lauroyl radicals are present in a molar ratio of 2:1, as in the substance trimethylolpropane dibenzoate monolaurate, forms a large amount of a crystalline precipitate after three weeks of storage at room temperature. In the course of storage at 4° C., the crystallization begins as early as after four days. C2 thus has insufficient storage stability and is therefore unsuitable as a plasticizer.

Surprisingly, and unforeseeably from the prior art, the inventive ester mixtures are notable in that they are liquids at room temperature and do not exhibit any tendency toward crystallization. As the comparison in Table 2 shows, the inventive ester mixtures, unlike the noninventive ester mixtures, are characterized by a distinctly lower crystallization tendency. Even after storage at 4° C. for over 60 days, the ester mixtures 1 to 4 remain clear and fluid.

Dissolution Temperature

The dissolution temperature in polyvinyl chloride is an important characteristic parameter for describing the gelling capacity of a pasticizer. Plasticizers having a dissolution temperature of above 170° C. are not economically viable since their processing demands too much energy. In addition, a dissolution temperature of above 170° C. indicates inadequate compatibility between plasticizer and polyvinyl chloride.

The inventive ester mixtures 1 to 5 have good gelling capacity. The comparative example C3, whose acid component consists predominantly of lauric acid, has a dissolution temperature of above 170° C. and is therefore unsuitable as a plasticizer.

Volatility

The volatility of the inventive ester mixture 2 and of the commercial plasticizer di(2-ethylhexyl) phthalate (“Vestinol® AH” from Oxeno Olefinchemie GmbH, abbreviation: DEHP) was determined with the aid of a Brabender H-A-G, E′ moisture tester by determining the weight loss in the course of heating of the plasticizer to 130° C. The weight loss is reported as a percentage based on the amount used.

TABLE 3
Volatility

| Experimental | | |
|---|---|---|
| duration, h | DEHP weight loss | Example 2 weight loss |
| 1 | 0.5% | 0.2% |
| 2 | 0.9% | 0.3% |
| 4 | 1.3% | 0.2% |
| 6 | 1.7% | 0.2% |

The ester mixture in Example 2 features lower volatility than the standard plasticizer di(2-ethylhexyl) phthalate.

Polyvinyl Chloride Compounds

For further testing, the ester mixtures 1 to 4 and the additives listed in Table 4 were used to produce the soft polyvinyl chloride compounds of the epoxy/Zn type (with epoxy-zinc stabilizer) and Pb type (with lead stabilizer).

TABLE 4
Composition of the polyvinyl chloride compounds

| | Parts in epoxy/Zn | Parts in Pb type |
|---|---|---|
| Constituent | type compounds | compounds |
| Polyvinyl chloride | 100 | 100 |
| (Vinnolit ® H70 DF) | | |
| Plasticizer | 70 | 70 |
| Stabilizer | 12 | 0 |
| (Crompton Mark ® EZ 735)¹⁾ | | |
| Stabilizer | 0 | 8 |
| (Interstab ® LGH 6017)²⁾ | | |
| Calcium stearate | 1 | 1 |
| Chalk (Omya ® BSH)³⁾ | 30 | 30 |
| Antioxidant | 0.2 | 0.2 |
| (Ciba ® Irganox ® 1010)⁴⁾ | | |

¹⁾Crompton Mark ® EZ 735 is an epoxy/Zn stabilizer.
²⁾Interstab ® LGH 6017 is a lead stabilizer from Akros Chemicals. According to the safety data sheet, it contains approx. 2% lead stearate (CAS No. 1072-35-1), approx. 6% dibasic lead stearate (CAS No. 12578-12-0) and approx. 83% dibasic lead phthalate (CAS No. 57142-78-6).
³⁾Omya ® BSH is a chalk from the Champagne region. The product is coated.
⁴⁾Ciba ® Irganox ® 1010 from Ciba Specialty Chemicals is a phenolic antioxidant having the formula pentaerythtrityl tetrakis(3,5-di-tert-butyl-4-hydroxycinnamate) (CAS No. 6683-19-8).

The components mentioned were first mixed at room temperature and subsequently rolled under the following conditions:

| | |
|---|---|
| Roller: | Servitec Polimix 110 L |
| Temperature: | 160° C. |
| Time: | 10 min |
| Roller speed of front roll: | 20 (rpm) |
| Roller speed of back roll: | 24 (rpm) |
| Thickness of the rolled sheet: | 0.7 mm |

The cooled rolled sheet was then pressed under the following conditions to give films:

| | |
|---|---|
| Press type: | Schwabenthan Polystat 200T |
| Temperature: | 170° C. |
| Time: | 10 min |
| Pressure: | 400 bar |
| Film thickness: | 0.3-1 mm |

Thermal Resistance of the Polyvinyl Chloride Compounds

The thermal resistance of the films was determined by the following test methods:

Storage in a Forced-Air Oven:

Films of size 30×30 mm with a thickness of 1 mm were stored hanging in a forced-air oven at the temperatures and times specified in Table 5. After the storage, the changes in weight were determined and reported in % based on the weight of the film used.

Congo Red Test:

The Congo Red test was carried out to DIN 53381-1 of 1971 using granule obtained from rolled sheets of thickness 0.7 mm. The time after which the colour change of the indicator is visible, occurring as a result of HCl release at 200° C., is listed in Table 5.

TABLE 5
Thermal resistance of the PCV compounds

| | | Forced-air | Forced-air | Forced-air | |
|---|---|---|---|---|---|
| | Forced-air oven | oven | oven | oven | |
| Test | 7 d/120° C. | 14 d/140° C. | 7 d/120° C. | 14 d/140° C. | Congo Red |
| Compd. type | Epoxy/Zn | Epoxy/Zn | Pb | Pb | Pb |
| Plasticizer | Change in | Change in | Change in | Change in | Min. before |
| | weight | weight | weight | weight | change |
| 1 | −1.8% | −4.2% | −1.7% | −7.9% | 215 |
| 2 | −1.4% | −3.6% | −2.6% | −7.1% | 233 |
| 3 | −1.7% | −4.7% | −1.9% | −7.3% | 241 |
| TOTM | −2.0% | −7.1% | −4.5% | −8.3% | 148 |

A high thermal resistance is expressed in a minimum weight loss in the forced-air oven and in a maximum time before change of the Congo Red indicator. The data in Table 5 demonstrate the better thermal stability on average of the inventive ester mixtures in comparison to trioctyl trimellitate.

Migration

In order to assess the migration of the inventive ester mixtures from soft polyvinyl chloride into another polymer, circular test specimens (Ø 50 mm) were produced from the above-described epoxy/Zn type polyvinyl chloride compound and contacted on both sides with polyethylene film (Atofina Laqtene® LDO 0304), and the contacted test specimens were stored in a drying cabinet at 70° C., weighted down with a 5 kg weight, and a change in weight of the test specimens was monitored over a period of 12 days. Table 6 reproduces the mean values of the changes in weight from a triple determination as % by weight based on the original specimen weights.

TABLE 6
Plasticizer migration

| Ester mixture/ | | | | |
|---|---|---|---|---|
| plasticizer used | 1 day | 2 days | 5 days | 12 days |
| 1 | −0.5% | −1.2% | −1.5% | −1.5% |
| 2 | −1.0% | −1.4% | −1.7% | −1.8% |
| TOTM | −1.3% | −1.8% | −2.3% | −2.5% |

The greater the weight loss measured, the greater the amount of ester mixture/plasticizer which has been transferred into the polyethylene by migration. The data in Table 7 demonstrate the distinctly better migration resistance of the inventive ester mixtures 1 and 2 in comparison to trioctyl trimellitate (TOTM).

Extraction

In order to assess the extraction of the inventive ester mixtures from soft polyvinyl chloride by liquid media, circular test specimens (Ø 60 mm) were produced from the above-described epoxy/Zn type polyvinyl chloride compound and immersed into a Petri dish filled with 50 ml of the media specified in Table 7, and the dishes were stored in a drying cabinet at the temperature specified in Table 7 for 10 days. Afterwards, the change in weight of the cleaned test specimens was determined and is reproduced in Table 7 as a % by weight based on the original sample weights.

TABLE 7
Plasticizer extraction

| | ASTM | ASTM | IRM | IRM | IRM | IRM |
|---|---|---|---|---|---|---|
| Ester mixture/ | oil | oil | 902 | 902 | 903 | 903 |
| plasticizer used | 23° C. | 60° C. | 23° C. | 60° C. | 23° C. | 60° C. |
| 1 | −2.5% | −17.0% | −2.2% | −15.9% | −2.9% | −13.2% |
| 2 | −3.5% | −18.2% | −2.6% | −15.8% | −3.8% | −13.1% |
| TOTM | −7.3% | −18.9% | −4.3% | −17.0% | −10.9% | −14.0% |

The larger the measured weight loss; the greater the amount of plasticizer which has been transferred into the medium by extraction. The data in Table 7 demonstrate the distinctly better extraction resistance of the inventive plasticizers 1 and 2 in comparison to trioctyl trimellitate.

What is claimed is:

1. An ester mixture containing
(A) 5-22% by weight of a compound of the general formula (I) 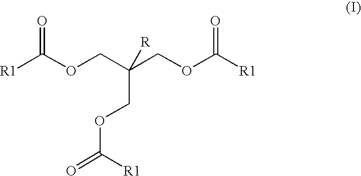
wherein
R is H or a C₁- to C₄-alkyl chain and
R¹ is a C₆- to C₁₄-aryl radical optionally substituted by one to three C₁- to C₄-alkyl radicals,
(B) 26-44% by weight of a compound of the general formula (II) 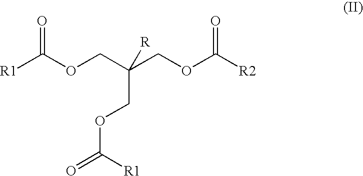
wherein
R and R¹ are each as defined above and
R² is a straight-chain or branched C₁₁- to C₂₁-alkyl radical,
(C) 28-45% by weight of a compound of the general formula (III) 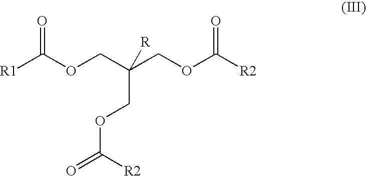
wherein
R, R¹ and R² are each as defined above and
(D) 6-25% by weight of a compound of the general formula (IV) 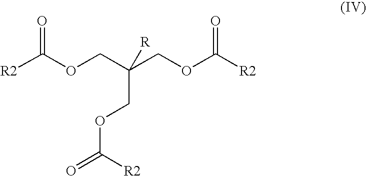
wherein
R and R² are each as defined above.

2. An ester mixture according to claim 1 wherein the R¹ radical derives from aromatic monocarboxylic acids.

3. An ester mixture according to claim 2 wherein such aromatic monocarboxylic acids are benzoic acid, o-toluic acid, nm-toluic acid, p-toluic acid, 4-tert-butylbenzoic acid, 1-naphthoic acid or 2-naphthoic acid or mixtures thereof.

4. An ester mixture according to claim 1 wherein the R² radical derives from aliphatic monocarboxylic acids.

5. An ester mixture according to claim 4 wherein such aliphatic monocarboxylic acids are lauric acid, myristic acid, palmitic acid, stearic acid, oleic acid, arachic acid or behenic acid or mixtures thereof.

6. An ester mixture according to claim 1 wherein the R radical derives from a tri-methylolalkane of the general formula (V) 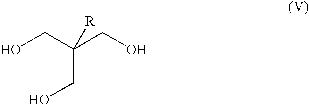
and
R represents H or a C₁- to C₄-alkyl chain.

7. An ester mixture according to claim 6 wherein the R radical derives from trimethylolalkanes.

8. An ester mixture according to claim 7 wherein the trimethylolalkane is trimethylolpropane or trimethylolethane.

9. A process for preparing the ester mixture according to claim 1 wherein
a) one or more trimethylolalkanes are esterified with
b) 108 to 180 mol % (based on 100 mol % of trimethylolalkane) of one or more aliphatic C₁₂- to C₂₂-monocarboxylic acids or aliphatic C₁₂- to C₂₂-monocarboxylic acid derivatives and
c) 120 to 300 mol % (based on 100 mol % of trimethylolalkane) of one or more aromatic C₇- to C₁₅-monocarboxylic acids or aromatic C₇- to C₁₅-monocarboxylic acid derivatives
d) at temperatures between 150° C. and 300° C.

10. A process according to claim 9, wherein such process is carried out
e) with the aid of catalysts and/or
f) with removal of volatile by-products of the esterification.

11. A process according to claim 9 wherein
the aromatic C₇- to C₁₅-monocarboxylic acids or aromatic C₇- to C₁₅-mono-carboxylic acid derivatives are be unsubstituted or C₁- to C₄-alkyl-substituted and/or
the aliphatic C₁₂- to C₂₂-monocarboxylic acids or aliphatic C₁₂- to C₂₂-monocarboxylic acid derivatives are be straight-chain or branched, saturated or olefinically unsaturated.

12. A process according to claim 9 wherein the aromatic C₇- to C₁₅-monocarboxylic acids or aromatic C₇- to C₁₅-monocarbobxylic acid derivatives are benzoic acid, o-toluic acid, m-toluic acid, p-toluic acid, 4-tert-butylbenzoic acid, 1-naphthoic acid and/or 2-naphthoic acid or mixtures thereof.

13. A process according to claim 9 wherein, the aliphatic C₁₂- to C₂₂-monocarboxylic acids or aliphatic C₁₂- to C₂₂-monocarboxylic acid derivatives are lauric acid, myristic acid, palmitic acid, stearic acid, oleic acid, arachic acid and/or behenic acid or mixtures thereof.

14. A process according to claim 9 wherein
trimethylolpropane is used as the trimethylolalkane;
benzoic acid is used as the aromatic C₇- to C₁₅-monocarboxylic acid; and
lauric acid is used as the aliphatic C₁₂- to C₂₂-monocarboxylic acid.

15. A method of use of the ester mixtures according to claim 1 as plasticizers for polymers such as polyvinyl chloride, vinyl chloride-based copolymers, polyvinylidene chloride, polyvinyl acetals, polyacrylates, polyamides, polylactides, cellulose and its derivatives, rubber polymers such as acrylonitrile-butadiene rubber, hydrogenated acrylonitrile-butadiene rubber, chloroprene rubber, chlorinated polyethylene, chlorosulphonyl polyethylene, ethylene-propylene rubber, acrylate rubber and/or epichlorohydrin rubber.

16. A Polymer comprising the ester mixtures according to claim 1.

17. A Polymer according to claim 16 wherein additives such as stabilizers, lubricants, fillers, pigments, flame retardants, light stabilizers, blowing agents, polymeric processing assistants, impact modifiers, optical brighteners, antistats and/or biostabilizers, and also mixtures thereof are comprised.

18. A Polymer according to claim 16 wherein monoalkyl esters of benzoic acid, benzoic diesters of mono-, di-, tri- or polyalkylene glycols, dialkyl esters of aliphatic diacids, dialkyl esters of aromatic diacids, trialkyl esters of aromatic triacids, phenyl esters of alkanesulphonic acids, alkyl or aryl esters of phosphoric acid, polyesters of dicarboxylic acids, and also mixtures thereof are comprised as further plasticizers.
